(19) United States
(12) Patent Application Publication
(10) Pub. No.: US 2024/0148508 A1
(43) Pub. Date: May 9, 2024

(54) Multi-Part Implant Having a Support Element and a Functional Element

(71) Applicant: Karl Leibinger Medizintechnik GMBH & Co. KG, Mühlheim/Donau (DE)

(72) Inventors: Adem Aksu, VS-Schwenningen (DE); Stefanie Grom, Neuhausen (DE); Frank Reinauer, Emmingen-Liptingen (DE); Tobias Wolfram, Dreieich (DE)

(21) Appl. No.: 18/547,209

(22) PCT Filed: Feb. 7, 2022
(86) PCT No.: PCT/EP2022/052836

§ 371 Date: Aug. 21, 2023

(30) Foreign Application Priority Data
Feb. 23, 2021 (DE) 10 2021 201 695.2

(51) Int. Cl. A61F 2/28

(52) U.S. Cl. CPC A61F 2/2846; A61F 2002/285; A61F 2210/0004; A61F 2220/0025; A61F 2250/006; A61F 2310/00017; A61F 2310/00023; A61F 2310/00041; A61F 2310/00083; A61F 2310/00179

(57) ABSTRACT

The invention makes available a multi-part implant (10), comprising:
a support element (20) for fixing the implant (10) to a bone material;
wherein the support element (20) forms a receiving space; and
a functional element (30) that can be introduced into the receiving space;
wherein the functional element (30) can be fixed in the receiving space (26) by the support element (20) at least with respect to one degree of freedom.

### TECHNICAL FIELD

The present invention relates to implants for bones, in particular those in which a certain hardness and rigidity of the implant is structurally required, while on the other hand good resorbability and/or penetrability of the implant for endogenous tissue is desired.

### BACKGROUND OF THE INVENTION

Today, bone defects can be treated using a variety of methods. Such bone defects occur, for example, during resections, after trauma, or as a result of bone inflammation or the like. Endogenous, autogenous bone transplantation is usually particularly preferred for the treatment. However, the availability of bone mass for such bone grafts is limited, and complications can arise at the donor site.

An alternative lies in biomaterials as bone replacement material, which have an osteoconductive effect, i.e. are able to act as a scaffold for natural bone growth. Particularly preferred biomaterials are those that are biocompatible and, after a healing time, have been replaced by regenerated new bone (i.e. new bone material).

However, implants are traditionally positioned using fixation elements such as screws or nails. This requires a basic initial strength of the implant or of the implant material. In the case of biomechanically weaker implant systems, e.g. made of resorbable ceramic or polymer materials, the initial strength may be a problem. Conventionally, this is solved by increased use of material and larger constructions. However, this solution is contrary to the objective of introducing as little foreign material as possible into a body and is limited in its applicability by geometric and stereotypical boundary conditions.

### SUMMARY OF THE INVENTION

The present invention addresses the problem of making available an improved implant. The problem is solved by an implant having the features of claim 1.

Accordingly, a multi-part implant (in particular a two-part implant) is provided which comprises a support element for fixing the implant to an organic hard tissue, in particular to bone material such as a plate bone or a tubular bone. A receiving structure of the support element forms a receiving space, in particular in such a way that the receiving space remains accessible to the outside (i.e. is not completely closed off). The implant also comprises a functional element that can be introduced (or is introduced) into the receiving space. The functional element can be fixed (or is fixed) in the receiving space by the support element (more precisely by the receiving structure of the support element) at least with respect to one degree of freedom.

The receiving structure is preferably designed in such a way that the functional element can be fixed or is fixed therein with respect to several, particularly preferably with respect to all, degrees of freedom. In some embodiments, it can be provided that the complete fixation with respect to all degrees of freedom of the functional element takes place only after the support element has been fixed to the bone material of the patient.

In particular, the basic idea of forming an implant in two parts (at least), namely with a support element and a functional element held and fixed by the latter, makes it possible to provide a certain rigidity and robustness of the implant, which is required, for example, for fixing the implant in/on the human body with a precise fit and secure against displacement, without the choice of material and structure for a functional part of the implant having to be restricted to any considerable extent. The functional element (or functional part) of the implant can be understood to mean the part that is intended to replace the body's own tissue, partially or completely, through biological, chemical and/or physical interaction, and/or that is to be resorbed by it.

It is therefore an idea of the present invention to provide the functional part of the implant through the functional element, while the support element fixes and/or stabilizes the implant, here in particular the functional element.

The support element thus compensates for the biomechanical disadvantages of the functional element of the implant, which tends to be softer and/or more brittle and which can also be referred to as an interactive and integrative “biocage”. In addition, when selecting the material for the functional element, its mechanical properties can largely be disregarded, so that the focus can be placed on the biologically and medically best and most promising material. This is because with the implant according to the invention, the mechanical stability of the implant as a whole is not provided, or is only barely provided, by the functional element, and instead rather comes from the support element. The support element thus provides additional options for the mechanical fixation and geometric arrangement and also stereospecific solutions for biocages (functional implant parts) that cannot otherwise be optimally implanted. The support element can also be referred to as a framework element

The implant can thus be designed in particular in such a way that the fixation of the implant takes place in a hard-tissue part of the patient (e.g. plate bone, long bone), while the biological activation of the functional element can take place in other tissue structures, preferably soft-tissue-like structures. The soft-tissue-like tissue structures (or soft tissue structures) include, for example, cartilage, muscle tissue or nerve tissue.

Depending on the desired application, both the support element and the functional element can be made of resorbable and/or non-resorbable materials. Both the support element and the functional element can each be made from a mixture of resorbable and non-resorbable materials. It is preferred that the support element is made of non-resorbable material and that the functional element is made at least partially, or completely, of resorbable material.

The functional element is particularly preferably porous, in order to facilitate blood flow through the functional element. By contrast, the support element is preferably solid (i.e. non-porous) in order to prevent any ingrowth of soft tissue into the support element. In this way, bone regeneration can be restricted to the desired geometry.

The invention can therefore be used particularly advantageously if the ideal, i.e. medically preferred, functional element is too brittle or too soft or for other reasons cannot be implanted directly or alone, e.g. because it cannot be fixed to/in the patient using the usual fastening means (screws, nails, adhesives, etc.) or cannot by itself maintain structural integrity. Therefore, in preferred material combinations of support element and functional element, in most embodiments the material of the support element is designed (or chosen) to be harder than the material of the functional element and/or less brittle than the material of the functional element.

The support element is also preferably made of a material that does not absorb any bone cells on the cover (protective cover) and can therefore be easily removed after bone regeneration has taken place. Thus, in some applications, after bone regeneration has taken place (or there is sufficient stability of the functional element interacting with the tissue), the support element can be extracted again from the body of the human or animal patient. In other applications, by contrast, provision can be made for the support element to remain implanted in the body even after (the best possible or most extensive or complete) resorption of the functional element has been completed. Leaving the support element in place may be useful in particular when its outer contour replaces the outer contour of a bone at a defect.

The support element can be fixed to the bone material using any desired fastening means, for example by means of (one or more) screws, pins, nails, wires, by means of suture material, and/or by means of adhesive.

Depending on the anatomical nature and purpose of use, it is also possible for several functional elements to be inserted into one and the same outer support element. Each functional element is preferably designed in one piece, particularly preferably produced by means of additive manufacturing. Alternatively, a functional element can also be composed of several parts, for example in order to accommodate a section of tissue between the two parts during the implantation of the implant. The outer support element can serve not only to fix the functional element as a whole, but also to fix the two (or more) individual parts of a functional element to one another (and thus possibly also a section of tissue enclosed between them).

According to some preferred embodiments, variants or developments of embodiments, the support element is designed as an outer support element and the receiving space is an interior space partially enclosed by the outer support element. “Partially enclosed” is intended to mean in particular that the interior is still partially open to the outside, in order to be able to accommodate the functional element. The receiving space or the interior space is preferably completely filled after introduction (or Insertion) of the functional element. In some embodiments, there is room for the whole of the functional element in the interior space; in other embodiments, the functional element protrudes beyond the interior space.

The design of the support element as an outer support element has the advantage that the support element can act, for example, as protection for the inner functional element (which in this case can also be designated as “inner element”) and can also fix the functional element from the outside and also hold it together structurally. In this way, it is also advantageous that the effects of high external forces on the functional element are avoided, such that bone regeneration can proceed without problems and without the functional element being displaced.

According to a number of preferred embodiments, variants or developments of embodiments, the support element has a window structure or a rib structure through which the functional element is accessible, in particular in addition to an opening through which the functional element can be introduced into the receiving space of the support element. In this way, tissue can interact more quickly and more easily with the functional element, and X-ray transparency of the implant can be improved. When inserted into the support element as intended, the functional element preferably closes the window structure or the rib structure. The window structure can have a frame that is closed all the way around, or it can also have only part of a frame that runs all the way round. In particular, an edge, or an edge portion, can be left free of the window structure, for example in order to provide access to tissue-receiving structures.

In other embodiments, the implantable support element can be designed such that it is positioned in the interior of the implant and fixes and/or structurally stabilizes the functional element from the inside.

The functional element can preferably be formed from a biocompatible, resorbable biomaterial. The functional element can, for example, be formed from tricalcium phosphate (p-TCP), hydroxyapatite (HA) or a biodegradable composite material (e.g. metal, polymer, ceramic or bioglass), which have a bone-like composition and good osteoconductivity. In some embodiments, the functional element consists of a biodegradable and biocompatible polymer mixed with calcium carbonate or magnesium. Likewise, specific embodiments can also be mixed with an apatite.

Generally possible materials for the functional element are all possible resorbable systems such as biodegradable magnesium, biodegradable magnesium alloys, biodegradable iron alloys, biodegradable zinc alloys, biodegradable ceramic systems, bioresorbable polymers or copolymers or hybrid variants or mixtures or combinations of the aforementioned materials.

Suitable biodegradable and biocompatible polymers include, for example: polyactide, e.g. PLLA (poly(L-lactic acid) or PDLLA (poly(D,L-lactic acid)) or PGA (polyglutamic acid) or PLGA (poly(lactide-coglycolide), a copolymer of the monomers lactide and glycolide) or PCL polycaprolactone).

According to a number of preferred embodiments, variants or developments of embodiments, the support element is formed from a non-resorbable material, in particular titanium or a titanium alloy, PEEK (polyetheretherketone), implant steel and/or UHMWPE (ultra-high-molecular-weight polyethylene, e.g. brand names Dyneema, IZANAS or Spectra).

According to a number of preferred embodiments, variants or developments of embodiments, the functional element can be clamped and/or clipped (or is clamped and/or clipped) into the receiving space. For this purpose, in particular, the receiving space can be formed by an inverted edge of the support element, with the functional element being able to be clamped into the inverted edge. In this way, the functional element can be fixed in relation to the support element without the use of additional fastening means, and it can then be fixed together with the support element, for example, to a bone of the patient.

The turned-back edge can have a curvature, such that the turned-back edge can fix or delimit the functional element on several sides or in several dimensions or degrees of freedom. There may also be several turned-back edges between which the functional element is initially clamped.

In these embodiments, it is particularly advantageous if the functional element is designed as a flat or plane structure with a thickness of 300-3000 micrometers, preferably between 500 and 2500 micrometers, particularly preferably between 900 and 1800 micrometers. Alternatively, the functional element can also be designed broader overall or with other shapes, but flatter at at least one edge, such that the functional element can be clamped or clipped with this edge into the protuberance (i.e. the receiving space) on the support element.

According to a number of preferred embodiments, variants or developments of embodiments, an interior of the functional element has at least one recess structure, which is designed to increase the surface area of the functional element, for example to enable the functional element to interact with the natural tissue of the patient over the largest possible area.

The at least one recess structure can comprise at least one cavity (preferably a plurality in each case) and/or at least one tunnel and/or at least one blind hole in the functional element. The dimensions of the at least one recess structure can be such that a capillary effect is created or suppressed.

Irrespective of the presence of one or more recess structures, it is moreover preferred that the functional element is porous in order to permit better blood flow (or penetration by other liquids such as lymphatic fluid, etc.).

According to a number of preferred embodiments, variants or developments of embodiments, the functional element has a tissue-receiving structure into which a tissue section can be inserted and/or through which a tissue section can be guided. There may be a single tissue-receiving structure with multiple openings to the outside, or multiple tissue-receiving structures. The support element advantageously has openings which correspond to the opening or openings of the tissue-receiving structure when the functional element is inserted into the support element. Such tissue parts (or tissue structures) can be, for example, blood vessels (arteries, veins, capillary vessels, etc.) or membranes. Tissue-receiving structures can be designed as trenches or channels open to the outside, for example in order to facilitate the introduction or embedding of tissue parts. However, tissue-receiving structures can also be closed on one side, for example if directed tissue ingrowth is to be achieved. In general, tissue-receiving structures that are open and closed on different sides can be provided, depending on what kind of tissue part is to be used or to grow in, from which side and up to where this is intended to take place, and further similar considerations.

According to a further aspect, the present invention also includes a method for implanting an implant according to the invention, comprising the steps of: inserting (in particular clamping or clipping) the functional element into the receiving space of the support element; introducing the support element, with the functional element inserted in the receiving space, into a human or animal body of a patient; fixing the support element to a bone in order to fix the implant. An optional step can include introducing a tissue section, or a tissue structure, of the patient into a tissue-receiving structure of the implant, it being possible for this tissue section to be introduced before, during and/or after the introduction of the outer support element into the patient. Another optional step may include removing the support element from the patient's body without removing the functional element from the patient's body.

### BRIEF DESCRIPTION OF THE FIGURES

The invention is explained in more detail below on the basis of exemplary embodiments in the figures of the drawing. In partially schematic representation:

FIG. 1 shows a schematic overview of a use of implants according to a first embodiment and according to a second embodiment;

FIG. 2 shows an assembled view of the implant from FIG. 1 according to the first embodiment, seen from the side;

FIG. 3 shows a cross-sectional view through the implant from FIG. 2;

FIG. 4 shows a view of the implant from FIG. 1 according to the second embodiment;

FIG. 5 shows a schematic representation of a top view of an implant according to a third embodiment of the present invention;

FIG. 6a shows a schematic side view of the implant from FIG. 5;

FIG. 6b shows a schematic cross-sectional view of the implant from FIG. 5.

In all of the figures, identical or functionally identical elements and devices have been provided with the same reference signs, unless indicated otherwise.

### DETAILED DESCRIPTION OF THE FIGURES

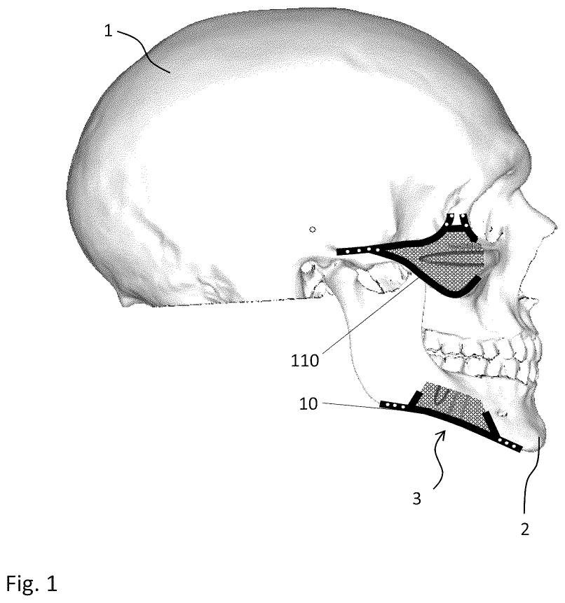

FIG. 1 shows a schematic representation of the use of an implant 10 according to a first embodiment of the present invention and of an implant 110 according to a second embodiment of the present invention.

FIG. 1 also shows a human skull 1. For the description of the implant 10, it is assumed that a piece 3 is missing from a lower jaw 2 of this skull, e.g. due to an accident or as a result of a resection that has been carried out. This means that in the present case the implant 10 is used for a plate bone. A defect on the right cheekbone of the skull 1 is assumed for the implant 110.

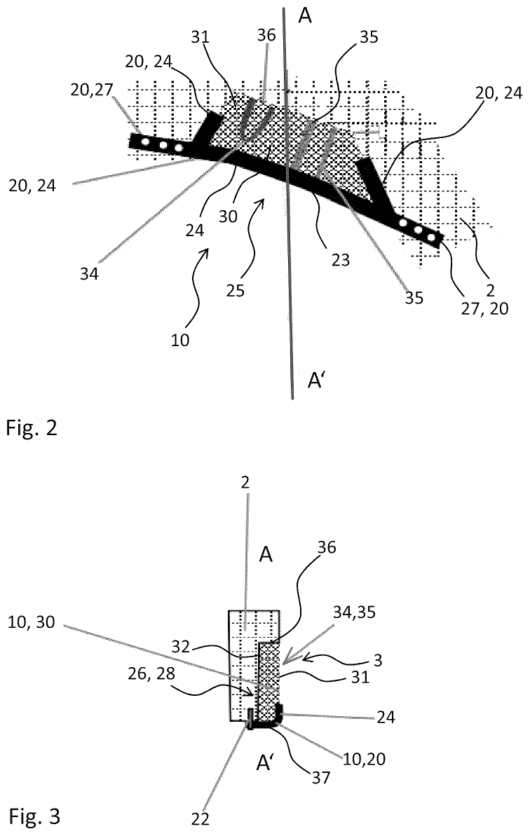

The implant 10 serves to replace the defect 3 in the lower jaw 2 with artificial material, at least in the short term. For this purpose, the implant 10 comprises an outer support element and a resorbable functional element, which is also explained in more detail below with reference to FIG. 2 and FIG. 3. The functional element is intended to promote and guide the natural bone regeneration and to be resorbed in regenerated bone tissue in the medium term.

FIG. 2 shows a view of the implant 10 from FIG. 1 seen from outside in the implanted state, the outside in this case meaning the side facing away from the skull 1 (or in general from the bone to be treated, replaced or supplemented) when the implant 10 has been implanted as intended.

It will be seen from FIG. 2 how the functional element 30 is inserted into the outer support element 20 and is already implanted together with the latter, i.e. in particular fixed to the bone (here the lower jaw 2).

The support element 20 can be formed, for example, from a non-resorbable material, in particular titanium or a titanium alloy, PEEK (polyetheretherketone), implant steel and/or UHMWPE (ultra-high-molecular-weight polyethylene, e.g. brand names Dyneema, IZANAS or Spectra).

The outer support element 20 is formed with an elongate carrier structure 23, at the two longitudinal ends of which are arranged fastening portions, in or on which fastening means, or aids for fastening means, are formed, here three screw holes 27 in each case. In addition to screw holes 27, other fastening means or auxiliary structures for fastening means (such as screw holes for screws) can of course also be formed on the outer support element 20. Hole structures for receiving pin systems, which can be used for temporary fixation, would also be possible.

Between the fastening portions of the carrier structure 23, a window structure 25 is formed as a further part of the outer support element 20, through which window structure 25 the functional element 30 (more precisely an outer side 31 of the functional element 30) can be seen and accessed in the non-implanted state. Thus, organic tissue, which covers the implant 10 after implantation, on the outside of the outer support element 20, can come directly into contact with the outside 31 of the functional element 30. In addition, such a window structure 25 can permit the greatest possible X-ray transparency of the implant 10.

The portion of the outer support element 20 around the window structure 25 may be referred to as frame structure 24. In the present case, the frame structure 24 is not completely closed around the window structure 25; in particular, it encloses the functional element 30, which is square in side view, completely only at one edge (through part of the carrier structure 23), and at two further edges only partially, preferably at least by half. The respective other part of these two further edges can be surrounded or touched by bone material, for example, as is shown more clearly in FIG. 3. At the fourth edge 36, the functional element 30 is delimited by the lower jaw bone 3 itself in the embodiment shown, as will be described in more detail below with reference to FIG. 3.

The frame structure 24 also encloses the functional element 30 at least partially on its outer side 31, in order to fix it in this direction, as will be described in more detail with reference to FIG. 3.

The resorbable functional element 30 can be made, for example, from biodegradable magnesium, from a biodegradable magnesium alloy, from a biodegradable iron alloy, from a biodegradable zinc alloy, from a biodegradable ceramic system, from a bioresorbable polymer or copolymer or hybrid variants or mixtures or combinations of the aforementioned materials.

The functional element 30 can have recess structures formed in the interior and/or on the surface of the functional element 30. The recess structures can in particular be at least one cavity (preferably in each case a large number of cavities) and/or at least one tunnel and/or at least one blind hole in the functional element 30. The dimensions of the at least one recess structure can be such that a capillary effect is created or suppressed.

The advantageous production of the functional element 30 by means of additive manufacturing allows these recess structures to be designed exactly according to plan and extremely precisely.

FIG. 2 also shows clearly that the functional element 30 optionally has a first tissue-receiving structure 34 and two further tissue-receiving structures 35.

As in the present case, these can be designed, for example, as a continuous trench, in particular in the outside 31 of the functional element 30, or as a continuous tunnel. In the present example, the first tissue-receiving structure 34 is designed as a continuous U-shaped trench, the longitudinal ends of which are both arranged on the same fourth edge 36 of the functional element 30. The further tissue-receiving structures 35 are designed as linear, straight trenches which extend from the fourth edge 36 of the functional element 30 to the opposite edge of the functional element 30, which edge bears on the outer support element 20 (more precisely on the frame structure 24 and the carrier structure 23) Each tissue-receiving structure 34, 35 can, for example, also be closed on one side, for example if directed tissue ingrowth is to be achieved.

A tissue part or a tissue structure of the patient can be introduced into the tissue-receiving structures 34, 35 after or during the implantation of the implant, in particular from the outside 31 and/or the edge 36 of the functional element 30. In this way, desired courses of this tissue part with respect to the implant 10 can be advantageously predetermined. The tissue part can be, for example, a blood vessel or a membrane or the like.

It will be appreciated that in other locations, or other anatomical circumstances, the tissue-receiving structures 34, 35 can also be designed as a blind hole with only a single opening, that the opening (or the openings) need not be arranged on an edge, and that an inlet opening and an outlet opening can be situated on different edges, and so on. These considerations apply to plane, flat functional elements 30 as in the present example. It will be appreciated that with other geometric shapes of the functional element 30, there are still many further possibilities for designing and arranging openings of the tissue-receiving structures 34, 35.

FIG. 3 shows a schematic cross-sectional view through the implant 10 from FIG. 2 along line A-A′. It will be seen clearly from FIG. 3 that the implant 10 in the present case is designed as an “onlay”, i.e. that the functional element 30 bears on a part of the bone (here the lower jaw 2). In the cross section in FIG. 3, it can be seen that the functional element 30 fills the defect 3 here. In other embodiments or applications, the functional element 30 can also be designed as a complete augmentation.

FIG. 3 also shows how the frame structure 24 can be curved in cross section, in order to partially enclose the functional element 30 at its edge 37, which completely covers the frame structure 24 (can be designated as “outer edge”), also on the outside 31 of the functional element 30. An inner side 32 of the functional element 30 opposite the outer side 31 is completely covered by the lower jawbone 2, and vice versa. This bending of the frame structure 24 in cross section thus forms a receiving structure 28 which defines a receiving space 26 for the functional element 30, wherein the receiving space 26 is completely filled by the functional element 30, but the functional element 30 (even for the most part) protrudes from the receiving space 26.

The frame structure 24 can also be curved in cross section at the two other further edges of the functional element 30, which are only partially enclosed by the frame structure 24, in order to also partially enclose the outer side 31 of the functional element 30 in each case.

A locking means 22 or a plurality of such locking means can be arranged on the edge 37 of the functional element 30, on the inside 32 of the functional element. Such locking means 22, for example a pin driven into the bone 2 through an opening in the outer support element 20, can also improve a connection (or fixation) of the outer support element 20 to (or on) the bone 2 in the region between the fastening portions at the longitudinal ends of the carrier structure 23.

The receiving space 26 serves to receive the functional element 30. Contours of the receiving space 26 are precisely adapted to the adjacent edges of the functional element 30. The receiving space 26 is preferably completely filled after the functional element 30 has been received.

The receiving pocket 28 can have one or more interruptions which allow access, from outside the implant 10, to an opening of at least one of the tissue-receiving structures 34, 35. These interruptions can also each be arranged where, according to a desired place of use or the local anatomy, the functional element 30 has openings.

In the embodiment shown, the receiving structure 28 is formed by turning back the frame structure 24 (more precisely by turning back the edge of the frame structure 24) of the outer support element 20 in the direction of the inside (i.e. in the direction of the bone 2), wherein, at the end of the turning back, the cross section of the frame structure 24 has substantially a 90 degree bend. This is advantageous in the present case since, in the onlay application, the healthy lower jaw bone 2 closes off (or forms) the receiving space 26 on the inside 32 of the functional element 30. In applications in which a defect in a bone plate is to be completely replaced (or filled) by the functional element 30, the turn-back can also be designed in such a way that it begins on the outside 31 of the functional element 30 and partially engages behind same, i.e. ends with a portion lying flat on an inside 32 of the functional element 30.

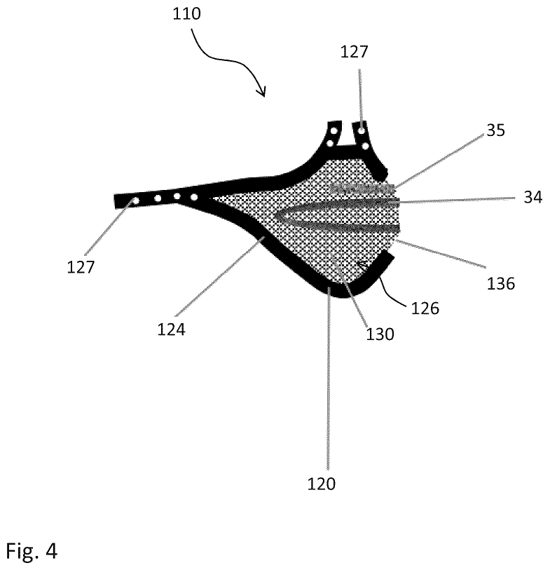

FIG. 4 shows a view of the implant from FIG. 1 according to the second embodiment. The implant 110 is a variant of the implant 10 and is also designed for a flat bone, as is evident in FIG. 1. In the case of the implant 110, an outer support element 120 in turn comprises a frame structure 124 which defines a receiving space 126 into which a resorbable functional element 130 can be inserted or is inserted.

As was described with reference to FIG. 3, the frame structure 124 can in each case be turned back in the direction of the bone in order to fix the functional element 130 laterally, or it can even turned back far enough to engage behind the functional element 130 on the inside thereof.

In this way, the turn-backs of the frame structure 124 form a receiving structure 128, which in turn defines a receiving space 126 for the functional element 130. In this case, the receiving space 126 is again completely filled by the functional element 130, which only protrudes to a very small extent (namely at the exposed edge section 136) from the receiving space 126, or optionally does not protrude at all. Depending on the type, position and degree of the turn-back, the receiving structure 128 fixes the functional element 130 in different directions or with regard to different degrees of freedom.

The outer support element 120 only partially encloses the functional element 130 on its outside (i.e. the side facing away from the bone), such that the functional element 130 remains accessible through the outer support element 120 on the outside. In other words, the frame structure 124 in turn forms a window structure through which the functional element 130 remains accessible to tissue lying on the implant 110.

The frame structure 124 encloses the functional element 130 almost completely, with an edge portion 136 of the substantially flat functional element 130 again remaining free. Openings of tissue-receiving structures 34, 35 can be formed on this free edge portion 136, as has already been described, for example, with reference to FIG. 2 and FIG. 3. Adapted to the bone geometry for which the implant 110 is designed, screw holes 127 for fixing the support element 120 are arranged on portions of the outer support element 120.

The outer support element 120, the functional element 130 and the fastening means can each be selected as described above. In particular, the support element 120 can be formed, for example, from a non-resorbable material, in particular titanium or a titanium alloy, PEEK (polyetheretherketone), implant steel and/or UHMWPE (ultra-high-molecular-weight polyethylene, e.g. brand names Dyneema, IZANAS or Spectra). The resorbable functional element 130 can be made, for example, from biodegradable magnesium, from a biodegradable magnesium alloy, from a biodegradable iron alloy, from a biodegradable zinc alloy, from a biodegradable ceramic system, from a bioresorbable polymer or copolymer or hybrid variants or mixtures or combinations of the aforementioned materials.

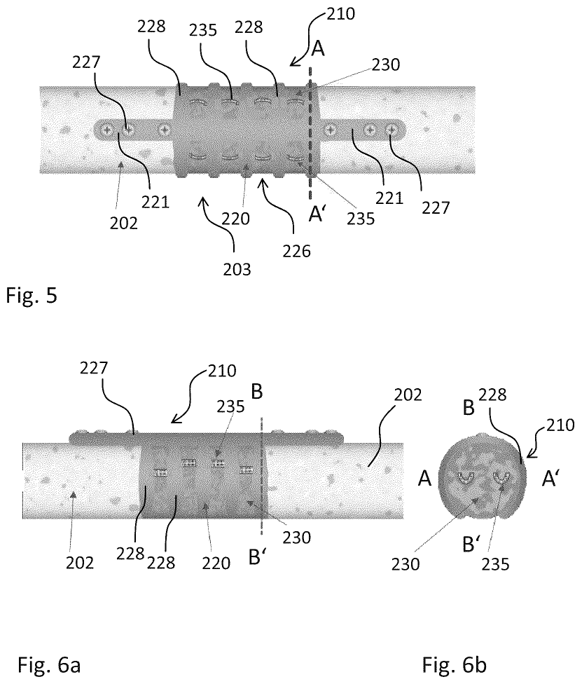

FIG. 5 shows a schematic representation of a top view of an implant 210 according to a third embodiment of the present invention. The implant 210 serves to correct, i.e. to fill, a defect 203 in a long bone 202. It often happens that material that is well suited for this purpose, for example on account of its resorption properties and the like, can be attached to the rest of the long bone 202 only with difficulty, and instead has to be fixed there until healing is complete and/or resorption has been carried out completely.

FIG. 6a shows the same situation as in FIG. 5, but from a side view; FIG. 6b shows a cross-sectional view along the section A-A′ in FIG. 5 or along the section B-B′ in FIG. 6a.

The implant 210 from FIG. 5 comprises an outer support element 220 and a functional element 230. The outer support element 220 comprises an elongate, straight web 221 (or an elongate carrier structure), which has connection means, here screw holes 227, at both ends. The two ends of the web 221 can each be fixed to a part of the tubular bone 202 by means of screws inserted through the screw holes 227. Ten rib structures 228 of the outer support element 220 are arranged on the web 221 in the region of the defect 203, the web 221 assuming a position with respect to the rib structures 128 similar to the human spine with respect to the human ribs. The rib structures 228 together enclose (or define) a cylindrical interior 226 (or receiving space).

The functional element 230 is also substantially cylindrical, i.e. as far as its outer contour is concerned, specifically in such a way that it can be introduced precisely into the cylindrical interior space 226. In the axial direction, the movement of the functional element 230 is restricted or prevented by the two parts of the long bone 202. In the tangential and radial directions, the movement of the functional element 230 is limited or prevented by the rib structures 228. The rib structures 228 thus form a receiving structure for the functional element 230.

It will be appreciated that long bones are not completely cylindrical in nature; accordingly, it will be appreciated that the implant 210 can be adapted to the actual shape of the long bone 202.

As will be seen particularly well from FIG. 6, the implant 210, with the functional element 230 inserted into the interior space 226, can be implanted very easily from above (in FIG. 6a), specifically in such a way that the interior space 226 with the functional element 230 exactly fills the defect 203. Bone screws can then be inserted into the screw holes 227, and the implant 210 can thus be screwed to the long bone 202 on one side, i.e. fixed to it.

The length of the web 221, the number of screw holes 227, the number of rib structures 228 and the like can each be adapted to the site of use.

FIG. 5, FIG. 6a and FIG. 6b also illustrate that the functional element 230 can again be formed with openings 235, which make a tissue-receiving structure (not shown) inside the functional element 230 accessible.

Although the present invention has been described above on the basis of preferred exemplary embodiments, it is not restricted thereto, and instead it is modifiable in a variety of ways. In particular, the invention can be altered or modified in many ways without departing from the core concept of the invention.

### LIST OF REFERENCE SIGNS

1 skull
2 lower jaw
3 defect
10 implant
20 outer support element
22 locking means
23 carrier structure
24 frame structure
25 window structure
26 interior space
27 screw holes
28 receiving structure
30 functional element
31 outside of the functional element
34 tissue-receiving structure
35 tissue-receiving structure
36 edge of the functional element
37 edge of the functional element
202 long bone
110 implant
124 frame structure
126 receiving space
127 screw holes
128 receiving structure
130 functional element
136 edge portion
203 defect 210 implant
220 outer support element
221 web
226 receiving space
227 screw holes
228 rib structures
230 functional element
235 openings

1. A multi-part implant comprising:
a support element configured for fixing the implant to a bone material;
wherein a receiving structure of the support element forms a receiving space; and
a functional element that is configured to be introduced into the receiving space;
wherein the functional element is configured to be fixed in the receiving space by the receiving structure at least with respect to one degree of freedom.

2. The multi-part implant as claimed in claim 1, wherein the support element is configured as an outer support element, and the receiving space is an interior space partially enclosed by the receiving structure.

3. The multi-part implant as claimed in claim 2, wherein the support element has a window structure or a rib structure through which the functional element in the receiving space is accessible.

4. The multi-part implant as claimed in claim 1, wherein the functional element is made of at least one of the following materials:
biodegradable magnesium;
biodegradable magnesium alloy;
biodegradable iron alloy;
biodegradable zinc alloy;
biodegradable ceramic system;
bioresorbable polymer or copolymer.

5. The multi-part implant as claimed in claim 1, wherein the support element is made from a non-resorbable material, from titanium or a titanium alloy, from polyetheretherketone, from implant steel and/or from UHMWPE (ultra-high molecular weight polyethylene).

6. The multi-part implant as claimed in claim 1, wherein the functional element is configured to be clamped and/or clipped into the receiving space.

7. The multi-part implant as claimed in claim 1, wherein the receiving space is formed at least by a turned-back edge of the support element.

8. The multi-part implant as claimed in claim 1, wherein an interior of the functional element has at least one recess structure which is configured to increase the surface area of the functional element.

9. The multi-part implant as claimed in claim 8, wherein the at least one recess structure comprises at least one cavity and/or at least one tunnel and/or at least one blind hole in the functional element.

10. The multi-part implant as claimed in claim 1, wherein the functional element has a tissue-receiving structure into which a tissue part can be inserted and/or through which a tissue part can be guided.
